(19) United States
(12) Patent Application Publication
(10) Pub. No.: US 2009/0124866 A1
(43) Pub. Date: May 14, 2009

(54) INTELLIGENT PERSONAL HEALTH PROFILE

(75) Inventors: NOEL J. GUILLAMA, WELLINGTON, FL (US); PEDRO MARTINEZ, BOCA RATON, FL (US); DONALD COHEN, BOCA RATON, FL (US); CHESTER HEATH, BOCA RATON, FL (US); DANIELLE AMODIO, WELLINGTON, FL (US)

Correspondence Address: COZEN O'CONNOR, P.C., 1900 MARKET STREET, PHILADELPHIA, PA, 19103-3508, US

(73) Assignee: THE QUANTUM GROUP, INC., WELLINGTON, FL (US)

(21) Appl. No.: 12/267,110

(22) Filed: Nov. 7, 2008

Related U.S. Application Data
(60) Provisional application No. 60/986,684, filed on Nov. 9, 2007.
(60) Provisional application No. 61/082,675, filed on Jul. 22, 2008.

(51) Int. Cl. A61B 5/00; G06G 7/48

(52) U.S. Cl. 600/300; 703/11

(57) ABSTRACT

A data processing system adapted to display a three dimensional representation of an individual patient's body, key functions, and current conditions, and to advise of options for improving wellness is disclosed. The system can be used, for example, to display a three dimensional image of a patient's body in a first state, to receive age and disease assumptions, and to display a three dimensional image corresponding to the received age and disease assumptions, thereby simulating the impact of lifestyle choices by representing the patient's body as it would appear at various ages having various diseases.

### CROSS-REFERENCE TO RELATED APPLICATIONS

Benefit of U.S. Provisional applications 60/986,684, filed Nov. 9, 2007, and 61/082,675, filed Jul. 22, 2008, is claimed. Both provisional applications are incorporated by reference in their entireties.

### BACKGROUND OF THE INVENTION

This invention is in the field of computer-assisted graphic representation of human bodies or parts thereof, more specifically to the field of computer graphics for medical applications.

The use of computer systems to display an image of a virtual patient is well known. For example, Haakonsen, U.S. Pat. No. 6,972,775, discloses a method of displaying an image of at least a portion of a virtual patient including accessing identification of a video file comprising data depicting virtual patient features over a range of the features, determining an offset into the video file, and presenting the video image corresponding to the offset. An example of medical simulation applications which reflect a virtual patient's weight over time when a user prescribes a diet, thereby displaying an appearance of the user's virtual patient is disclosed.

Attar, et al., U.S. Pat. No. 6,734,858, disclose a method and system for progressively aging an image for a first predetermined period of time and a second predetermined period of time and outputting the resultant progressively aged images to show an individual the personalized benefits that they might expect to realize using a personal care product.

Blanz, et al., U.S. Pat. No. 6,556,196, disclose a method of providing a morphable object model derived from a plurality of 3D images, matching the morphable object model to at least one 2D object image, and providing the matched morphable object model as a 3D representation of the object. For example, 3D faces are constructed from 2D photographs of a faces.

Lobo, et al., U.S. Pat. No. 5,781,650, disclose a process for automatically finding facial images of a human face in an electronically digitized image, e.g., taken by a video camera, and classifying the age of the person associated with the face into an age category.

Murata, et al., U.S. Pat. No. 5,818,457, disclose a face image data processing device which creates a face image suitable for an age and presumes the age of the created face using an age presumption module.

Bravomalo, U.S. Pat. No. 6,643,385, discloses a virtual fitness planner which combines image morphing technology, exercise programming, supplement sales, and motivational techniques into one product wherein users enter their current measurements, measurement goals, and current picture into the system preferably via a Web site. The picture is segmented into body components and each segment is morphed based on the goal measurement and the segment's responsiveness to weight loss using physiological tables and formulae. The resultant modified image is returned to the user over the internet.

McAlpine, et al., U.S. Pat. No. 7,209,577, disclose a facial feature localized and global real time video morphing system.

Linford, et al, U.S. Pat. No. 5,825,941, disclose an aesthetic image system for use in editing digital images.

It is clear from the prior art that there is strong interest in presenting images of users such as patients via a computer system and often over the internet, and there is interest in presenting the effect on a person of changes in lifestyle such as weight management programs and vitamin supplements, no one has suggested a system or method wherein a three dimensional representation of an individual patient's body, key functions, and current conditions, are displayed along with advise as to options for improving wellness, especially based on one or more health models based on data from a population of a plurality of individual patients.

It is an object of the present invention to provide a system and method for simulating the impact of lifestyle choices by representing or simulating the body as it would appear at various ages having various diseases. It is another object of the invention to provide a system for simulating the impact of lifestyle choices by representing the patient's body as it would appear at various ages having various diseases.

### SUMMARY OF THE INVENTION

These objects, and others which will become apparent from the following detailed description, are achieved by the present invention which comprises in one aspect a data processing system adapted to display a three dimensional representation of an individual patient's body, key functions, and current conditions, and to advise of options for improving wellness.

In another aspect, the invention comprises a data processing system adapted to display a three dimensional representation of an individual patient's body in a first, current state, to receive age and disease assumptions, and to display one or more three dimensional images in at least a second state, corresponding to the received age and disease assumptions related to lifestyle choices, thereby simulating the impact of lifestyle choices by representing the patient's body as it would appear at various ages having various diseases.

Preferably the display is calculated from health data for the individual patient and one or more health models based on data from a population of a plurality of individual patients. A plurality of functions and conditions of the patient's body can be analyzed and the options for improving wellness can be advised on the display in preferred embodiments. It is also preferred that the three dimensional representation is calculated from a plurality of sources of patient condition data. Current medication and therapy status are displayed in some embodiments. The system and method of the invention can advise of options for improving wellness which are calculated based on relationships between a plurality of patient symptoms, for example wherein the relationships are based on patterns determined by processing a statistically significant number of patient records, based on a statistically significant sample size.

The invention can simulate on a three dimensional representation of an individual patient's body both normal age progressions and age progressions assuming one or more specific diseases related to lifestyle choices for the purpose of simulating the impacts of lifestyle choices.

The invention can be used for clinical and patient education in some embodiments and can simulate the effect of assumed lifestyle improvements including in some embodiments on multiple alternative assumptions shown simultaneously, where more than one lifestyle initiated aging factor may exist. The lifestyle choices can be any of a wide variety, including any known now or in the future to affect a person's appearance, for example smoking, exercise, cholesterol medication, obesity, sun exposure, sunscreen use, weight control program, yoga, meditation, alcoholism, level of alcohol consumption, cocaine use, heroin use, stress, eating disorder, anorexia, bulimia, and marijuana use.

### BRIEF DESCRIPTION OF THE DRAWINGS

FIG. 1 is a representation of a displayed set of images of a patient in a current state and in various states resulting from alternative lifestyle choices.

FIG. 2 is a representation of an input screen on a computer display wherein various options are presented.

FIG. 3 is a table showing textually the results of lifestyle alternatives at a selection of various stages.

FIG. 4 is a representation of a display of a plurality of body part and organs of a patient wherein the effect of lifestyle choices can be displayed for each body part or organ.

FIG. 5 is a representation of a display wherein skeletal images of an individual with simulated disease states are presented.

FIG. 6 is a schematic of a system of the invention.

### DETAILED DESCRIPTION

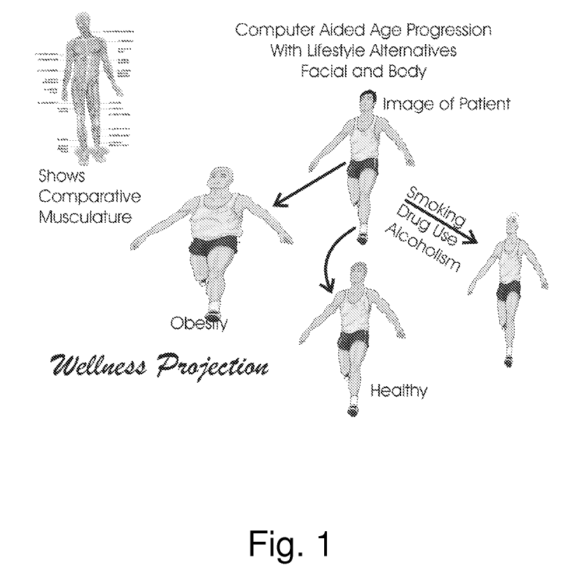

While the invention is capable of a wide range of embodiments and can be implemented with a wide range of software and hardware choices, a few illustrative embodiments will be described below wherein FIG. 1 is a wellness projection which includes the effects of lifestyle choices on appearance of the body. According to the invention a three-dimensional computer graphic representation is used to illustrate the human body as a multi-faceted and dynamic living entity. The conditions, treatments and developments make this model unique to each individual. For the purpose of patient and physician education, the invention enables visualizing both the external and internal effect of future lifestyle patterns and changes on the body. The means combines age-progression software and morphing toward specific physical appearance directions accelerated by disease patterns to yield a “malady-map” or displayable sequence of progressive stages as, for example, shown in FIG. 1. For most individuals the progression of age appearance can be modeled by software to compress or elongate portions of the face during childhood, or to emphasize facial wrinkles and expression lines along with growth of soft tissue, or skin conditions after the skull is fixed. These effects of aging are most pronounced in the face, but whole body progression with obesity, osteoporosis, skeletal changes with posture and deteriorating musculature can also be simulated. The system can selectively age an individual according to various lifestyle options including but not limited to smoking, exercise, cholesterol medication, obesity, sun exposure, sunscreen use, weight control program, yoga, meditation, alcoholism, level of alcohol consumption, cocaine use, heroin use, stress, eating disorder, anorexia, bulimia, and marijuana use. Each lifestyle has specific deleterious patterns. Elective lifestyle has been shown to contribute more than 50% to disease processes as we age.

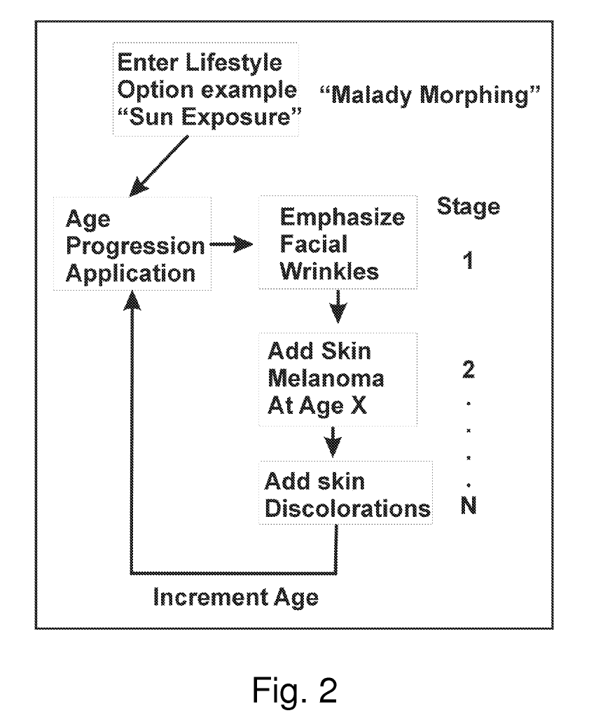

Referring now to FIG. 2, the system can be used to educate a patient that sun exposure leads to wrinkling, skin deterioration, discoloration and potential melonoma according to a predictable schedule. Similarly but not illustrated, obesity can change the shape of the face and body in predictable patterns and potentially leads to diabetes, with attendent shin problems as well.

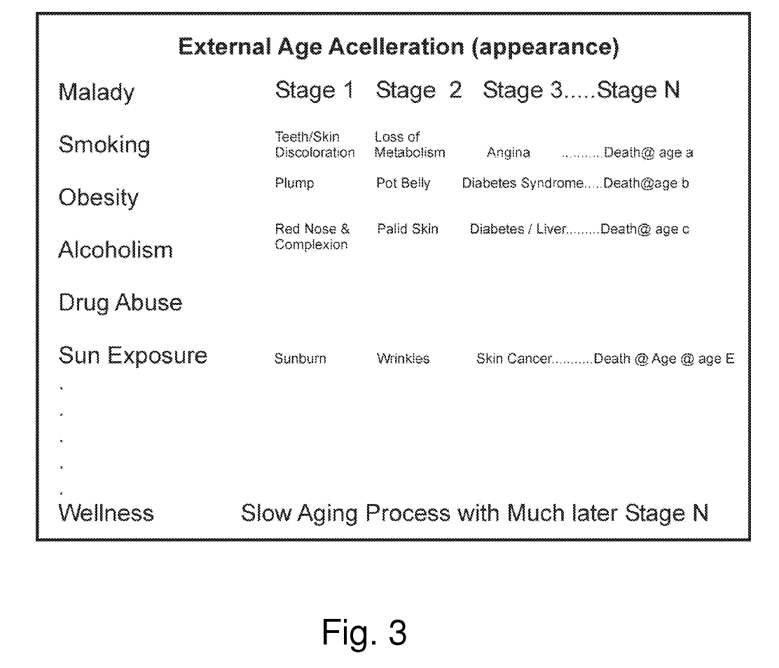

A table such as shown in FIG. 3 of accelerated aging characteristics can be used to bias age progression algorithms to present these changes to the individual as part of a program to alter lifestyle or as a clinical teaching tool.

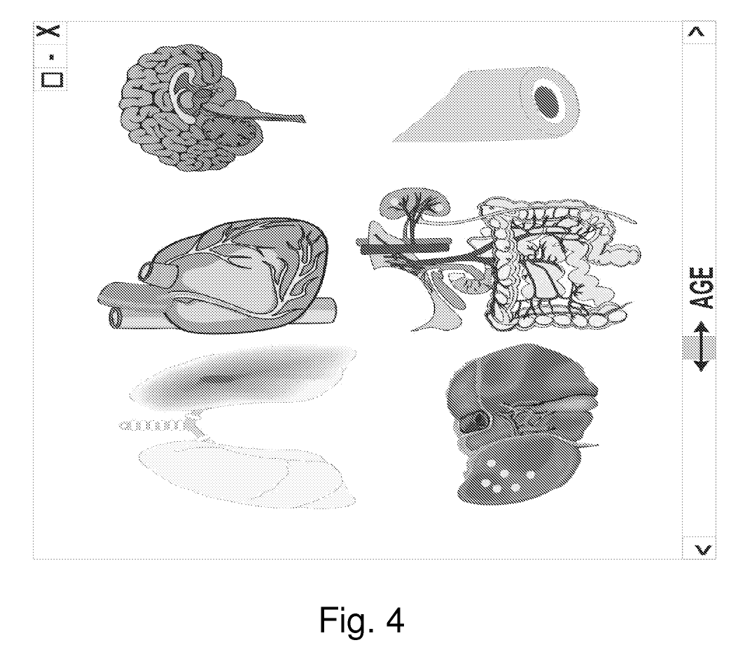

In a similar fashion, as shown in FIG. 4, the impact of disease on internal organs, with progression as disease and aging advance can also be simulated. The progression of lung cancer, arterial sclerosis, and lesions of intestine and liver are shown. With some predictability, these maladies can be shown to arrive on advanced schedules with poor lifestyle. The system and method provide a diagram showing an age progression slider on the right side, where the slow advancement can be depicted to occur at relative age.

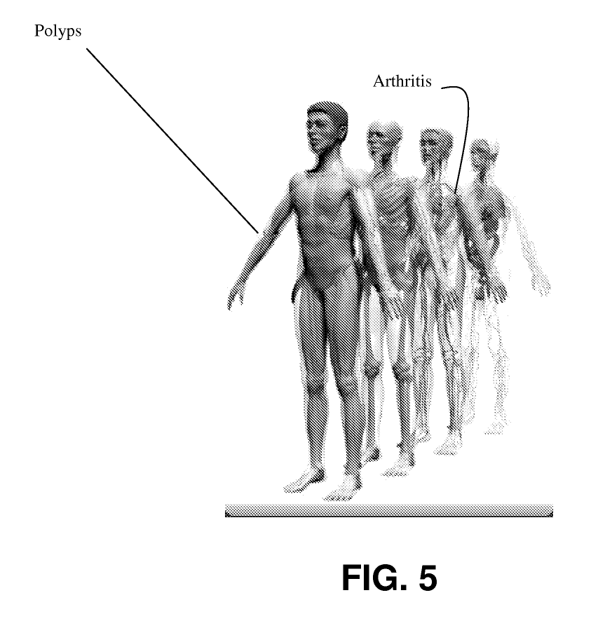

In FIG. 5 a three-dimensional computer graphic representation is used to illustrate the human body as a multi-faceted and dynamic living entity. The conditions, treatments and developments make this model unique to each individual. Multiple data sources are preferably aggregated to define the current state of the different bodily functions. Polyps in the colon can be highlighted as part of the digestive system. Arthritis can be isolated as part of the skeletal structure. These data sources provide the first element of analysis; presentation. These different current states can be overlaid first with current care (medication, therapy), then they can be further enhanced by recommending additional long-term wellness programs (diet, exercise). More in-depth analysis and recommendation can be invoked by computer linking to related articles, documents and experts. This real-time model can be used by both the individual and care provider to assess the full body condition (symptoms, observations, medications, treatments). A new dimension of understanding the total body condition is realized when multiple disparate symptoms are analyzed in conjunction (i.e. relationship between gum disease and heart malfunctions). This level of artificial intelligence can anticipate and project care. By providing a visual representation, the individual and provider can both understand and plan. This expert system is dynamic and constantly updated by changes to the individual's record.

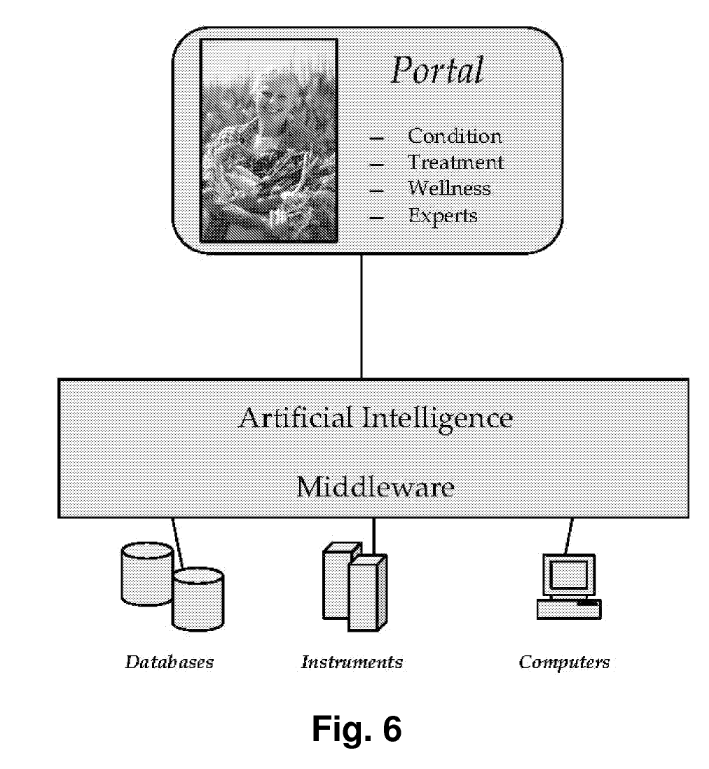

Referring now to FIG. 6, three layers of technology are integrated to achieve the visual and interactive model. The first is the user “portal” that provides the visual presentation and interactivity. The second is the middleware that provides the links to the multiple sources of data. The third and critical to synthesis of data is the artificial intelligence engine that searches through millions of records, analyzes for related patterns and presents recommendations based on extensive information sources. FIG. 6 highlights the three layers of technology in an integrated model.

According to one embodiment of the method aspect of the invention, an individual accesses the Web and links to their “Personal Health Profile” in a private and secure environment.

The user and doctor jointly analyze the conditions of the cardio vascular system by clicking on the heart. The image reveals the latest x-rays, catheterization, EKG and vitals. The doctor clicks again and views the lab results of cholesterol and lipids. The doctor then clicks on the treatments button and Lipitor 20 mg/day is displayed. Based on age, cholesterol levels and medications the “wellness” Button is hit and a series of recommended dietary plans, exercise routines and subsequent check ups are recommended. This includes related articles and links to experts (e.g., nutritionists). The doctor now hits the “artificial intelligence” button. At this time, the current exam is synthesized with all other conditions. The “artificial intelligence” engine highlights an entry by the patient describing pain in the joints and muscles; it further flashes the potential side effects of Lipitor being muscle and joint pain (a serious condition). With this information, the doctor changes the prescription to Crestor 10 mg. He schedules lab tests at 30 days. The lab tests are automatically scheduled into the patient's electronic schedule. The doctor asks the patient “anything else”? Shows the patient a listing of the tests done, the charges and how much the insurance covers. The patient provides his electronic signature and the record is sent electronically to the insurance company, the doctor's records and the patient's “Personal Health Profile” An entirely new perspective of a patient's profile can be derived from the integration of an individual's conditions, treatments and genomic code. Computer Artificial Intelligence methods will provide the analysis, synthesis and presentation of these complex integrated profiles of the human body.

As seen in the illustrated embodiments, a data processing system adapted to display a three dimensional representation of a patient's body, key functions, and current conditions, and to advise of options for improving wellness is demonstrated. A comprehensive representation of an individuals' “Personal Health Profile” is created through the integration of a user metaphor, health records and intelligence agents (artificial intelligence). A three-dimensional computer model provides a visual representation of: a person's body, key functions (i.e. cardio vascular, digestive, muscular), their current condition and recommendations for optimum wellness. The uniqueness and novelty of the invention is in the combination of multiple sources of data (x-ray, cat scan, doctor notes, vitals) into an advanced model for integration of virtual data, interactive presentation and derived intelligence in real-time. This provides the collection, analysis and synthesis of disparate information into an interactive, visual and understandable model for individual wellness and care.

A an important aspect of preferred embodiments of the invention is the analysis of the total body simultaneously; instead of separate functions, and through artificial intelligence uncover and understand trends and behavior of the total body as a system. The most advanced feature of this invention is the link and synthesis of an individual's profile, conditions and treatments in context with their genetic code. This invention provides for the integration of relational data; inclusive of medical, geographic and lifestyle with genetic data. Both of these factors are aggregated with a much larger relevant population mix in order to derive statistically valid sampling, contextual analysis and projection. The extraction of trends from the integration, synthesis and analysis of data can provide the healthcare and pharmaceutical industries with unprecedented insight about real life conditions of multiple symptoms, multi-drug interactions and post clinical trial results.

The present invention, therefore, is well adapted to carry out the objects and attain the ends and advantages mentioned, as well as others inherent therein. While the invention has been depicted and described and is defined by reference to particular preferred embodiments of the invention, such references do not imply a limitation on the invention, and no such limitation is to be inferred. The invention is capable of considerable modification, alteration and equivalents in form and function, as will occur to those ordinarily skilled in the pertinent arts. The depicted and described preferred embodiments of the invention are exemplary only and are not exhaustive of the scope of the invention. Consequently, the invention is intended to be limited only by the spirit and scope of the appended claims, giving full cognizance to equivalents in all respects.

What is claimed is:

1. A data processing system adapted to display a three dimensional representation of an individual patient's body, key functions, and current conditions, and to advise of options for improving wellness.

2. The system of claim 1 wherein the display is calculated from health data for the individual patient and one or more health models based on data from a population of a plurality of individual patients.

3. The system of claim 1 wherein a plurality of functions and conditions of the patient's body are analyzed and the options for improving wellness are advised on the display.

4. The system of claim 1 wherein the three dimensional representation is calculated from a plurality of sources of patient condition data.

5. The system of claim 1 wherein current medication and therapy status is displayed.

6. The system of claim 1 wherein the advice of options for improving wellness is calculated based on relationships between a plurality of patient symptoms.

7. The system of claim 1 wherein the advice of options for improving wellness is calculated based on relationships between a plurality of patient symptoms, and wherein the relationships are based on patterns determined by processing a statistically significant number of patient records.

8. The system of claim 1 wherein the advice of options for improving wellness is calculated based on relationships between a plurality of patient symptoms, and wherein the relationships are based on patterns determined by processing a statistically significant sample size.

9. A data processing system adapted to display a three dimensional representation of an individual patient's body in a first, current state, to receive age and disease assumptions, and to display one or more three dimensional images in at least a second state, corresponding to the received age and disease assumptions related to lifestyle choices, thereby simulating the impact of lifestyle choices by representing the patient's body as it would appear at various ages having various diseases.

10. The data processing system of claim 9 adapted to simulate on the three dimensional representation of an individual patient's body both normal age progressions and age progressions assuming one or more specific diseases related to lifestyle choices for the purpose of simulating the impacts of lifestyle choices.

11. A data processing system according to claim 9 adapted for clinical and patient education.

12. The data processing system of claim 9 adapted to simulate the effect of assumed lifestyle improvements.

13. The data processing system of claims 9 adapted to simultaneously display images based on multiple alternative assumptions shown simultaneously, where more than one lifestyle initiated aging factor may exist.

14. The data processing system of claims 9 wherein the lifestyle choices are selected from the group consisting of smoking, exercise, cholesterol medication, obesity, sun exposure, sunscreen use, weight control program, yoga, meditation, alcoholism, level of alcohol consumption, cocaine use, heroin use, stress, eating disorder, anorexia, bulimia, and marijuana use.

15. The data processing system of claim 9 wherein advice for improving wellness is displayed.

16. A method of advise of options for improving wellness comprising providing a data processing system adapted to display a three dimensional representation of an individual patient's body, key functions, and current conditions.

17. The method of claim 16 wherein a three dimensional representation of an individual patient's body in a first, current state, is displayed, age and disease assumptions are inputted, and one or more three dimensional images in at least a second state are displayed, corresponding to the received age and disease assumptions related to lifestyle choices, thereby simulating the impact of lifestyle choices by representing the patient's body as it would appear at various ages having various diseases.
